(19) United States
(12) Patent Application Publication
(10) Pub. No.: US 2019/0022257 A1
(43) Pub. Date: Jan. 24, 2019

(54) ULTRAFINE NANOPARTICLES AS AN IMAGING AGENT FOR DIAGNOSING A RENAL DISORDER

(71) Applicants: INSTITUT NATIONAL DE LA SANTE ET DE LA RECHERCHE MEDICALE (INSERM), Paris (FR); UNIVERSITE CLAUDE BERNARD LYON I, Villeurbanne (FR); UNIVERSITE DE PARIS 5 RENE DESCARTES, Paris (FR); CENTRE NATIONAL DE LA RECHERCHE SCIENTIFIQUE, Paris (FR); ECOLE NATIONALE SUPERIEURE DE CHIMIE DE PARIS, Paris (FR)

(72) Inventors: Nathalie MIGNET, Clamart (FR); Bich-Thuy DOAN, Carrieres sur Seine (FR); Grégory RAMNICEANU, Paris (FR); Olivier TILLEMENT, Fontaines Saint Martin (FR); Lucie SANCEY GALLIOT, Paladru (FR); François LUX, Lyon (FR); Johanne SEGUIN, Le Kremlin-Bicetre (FR)

(21) Appl. No.: 16/069,728

(22) PCT Filed: Jan. 13, 2017
(86) PCT No.: PCT/EP2017/050682

§ 371 Date: Jul. 12, 2018

(30) Foreign Application Priority Data
Jan. 15, 2016 (EP) 16305038.8

(51) Int. Cl. A61K 49/12; A61K 49/18; A61K 51/12; A61K 51/06

(52) U.S. Cl. CPC A61K 49/128; A61K 49/1881; B82Y 5/00; A61K 51/065; A61K 2123/00; A61K 51/1244

(57) ABSTRACT

The invention relates to a novel use of ultrafine nanoparticles as an imaging agent in a method for diagnosing a renal disorder. The invention also relates to the use of ultrafine nanoparticles as an imaging agent in methods for monitoring the therapeutic efficacy of a renal disorder treatment.

### TECHNICAL Field

The invention relates to a novel use of ultrafine nanoparticles as an agent for diagnosing a renal disorder.

### TECHNICAL Background

The kidney is an organ which has the function of purifying the blood. It can thus filter up to 170 liters of blood per day in human beings. By filtering the blood, the kidney produces urine. Urine is a mixture composed of water, mineral salts (sodium, potassium, calcium, etc.) and toxic waste such as urea and creatinine.

In addition to the filtration of blood and the production of urine, the kidney also performs other important physiological functions, such as the regulation of water and salt content in the body, the production of erythropoietin, which is essential for the formation of red blood cells, the production of the active form of vitamin D which is involved in maintaining blood calcium content, the regulation of body fluid composition or the production of renin which is involved in blood pressure regulation.

Pathological renal conditions can be of various types and result in a renal disorder. Renal disorder generally results in a decrease in renal functionality, in particular a decrease in tubular function and/or a decrease glomerular filtration. It can go as far as renal failure in the most serious cases.

Renal failure corresponds to the impairment of kidney function, said kidneys no longer correctly filtering the blood. The disease is said to be acute if the disorder is transient and reversible, and chronic if the destruction is irreversible, without the possibility of recovery. If the renal failure is major, the renal function can be replaced by dialysis or transplantation. Dialysis makes it possible to filter the blood via a derived circuit, usually outside the body.

Acute renal failure usually occurs after an attack such as an abrupt and transient decrease in blood pressure, during a hemorrhage, a general infection (septicemia), a drug intoxication or else in the event of obstruction of the urinary tracts by a kidney stone or a prostate adenoma. The kidneys take a few days to spontaneously return to normal function after treatment. During this period, it is sometimes recommended to use dialysis, which allows the patient to survive during the renal self-repair process.

Chronic renal failure by definition does not regress. It can be induced by pathological conditions (diabetes, hypertension, etc.) which gradually and irreversibly destroy the various renal structures. There are five stages of the disease up to the end stage during which the filtration capacity is less than 15% of normal for the kidneys as a whole. This stage requires envisioning techniques for replacing renal function: dialysis and/or transplantation.

Individuals affected by renal failure can remain in apparent good health with kidneys operating at from 10 to 20% of their normal capacity. This is because the main symptoms of renal failure only show up at an advanced stage, making it difficult to diagnose the disease. It is nevertheless important to act early in order to avoid the complications associated with renal failure and/or to prevent the progression of renal failure.

According to a study carried out in the United States over the period 1999-2000 (National Health and Nutrition Examination Survey IV), 9.4% of the general population, two thirds being men, appear to exhibit renal failure, including 5.6% at a slight or moderate stage, 3.7% at a severe stage and 0.13% at an end stage (Coresh J, Byrd-Holt D, Astor B C, et al. Chronic kidney disease awareness, prevalence, and trends among U.S. adults, 1999 to 2000. J Am Soc Nephrol 2005;16:180-8). The disease is rare before the age of 45, but its prevalence increases with age, in particular after the age of 65. The prevalence of this disease should further increase in the coming years owing to the aging of the population and the increase in diabetes, two major causes of renal failure, and through the improvement in survival of patients having received a transplant and of patients receiving dialysis.

Renal failure can be the result of renal fibrosis. Renal fibrosis is due to an excessive accumulation of extracellular matrix in the renal parenchyma. It results from a destabilization of a complex balance between profibrotic and antifibrotic cells and proteins. The precise mechanisms regulating the occurrence of fibrosis have at the current time only been incompletely elucidated and for the moment there is no effective treatment for fibrosis which allows a return to a normally functioning renal parenchyma once the disease has taken hold. Thus, renal fibrosis results more or less rapidly in chronic end-stage renal failure.

It is therefore essential to be able to efficiently diagnose renal disorder in order to treat patients as quickly as possible and thus to prevent the progression of the disease, in particular the progression toward chronic renal failure.

Generally, renal disorder is diagnosed by means of urine and/or blood tests. The markers to be taken into account may be of various natures. For example, the Haute Autorité de Santé (HAS) [French National Health Authority] recommends evaluating renal function on the basis of creatinemia, by estimating the glomerular filtration rate (GFR) and assaying creatinine using an enzymatic method.

Nevertheless, the assaying of creatinine is not sufficient since there is no linear relationship between creatinemia and glomerular filtration (renal filtration). Furthermore, blood and/or urine tests do not always make it possible to provide a precise diagnosis of renal disorder, in particular of renal failure. This is because these tests are merely elements which make it possible to point the physician in a direction, and may reveal other pathological conditions. In order to confirm renal disorder, it is often necessary to perform other tests, or even to seek advice from specialists such as a nephrologist, a urologist or a cardiologist. The diagnosis may therefore be lengthy and expensive, since it requires the performing of several tests and the consultation of various specialists.

Moreover, for some types of patients, it may be recommended to carry out other tests, for instance a search for microalbuminuria in type 1 and 2 diabetic patients.

Imaging techniques may also be used to diagnose renal disorder, for instance radiology, ultrasound, tomodensitometry (TDM) or magnetic resonance imaging (MRI). These techniques are generally used to identify the causes of renal disorder.

There is therefore a real need to precisely and reliably diagnose any type of renal disorder.

The inventors have shown that ultrafine nanoparticles, having an average hydrodynamic diameter of less than 10 nm, advantageously less than 5 nm, for example ranging from 1 to 5 nm, can be used as an imaging agent that is particularly favorable for imaging the kidney. These nanoparticles make it possible to obtain reliable and precise information on renal function, in particular by MRI imaging. Thus, these ultrafine nanoparticles are particularly advantageous for use in a method for diagnosing, by imaging, a renal disorder, such as renal fibrosis and/or renal failure.

### SUMMARY OF THE INVENTION

The present invention aims to provide novel imaging methods, in particular by MRI, allowing the reliable detection of a renal disorder, in particular renal fibrosis and/or renal failure, optionally coupled to a therapeutic procedure.

According to a first aspect, the invention relates to nanoparticles intended to be used as an imaging agent in a method for diagnosing, in vivo, a renal disorder in human beings or animals, said nanoparticles comprising:
(i) a polyorganosiloxane (POS) matrix;
(ii) one or more chelating agents, preferably from 6 to 20 chelating agents, grafted onto the POS matrix by —Si—C— covalent bonding;
(iii) one or more metal ions chelated by one or more of the abovementioned chelating agents.

According to a second aspect, the invention relates to the use of the nanoparticles as defined above, as an imaging agent in the in vivo diagnosis of a renal disorder in human beings or animals.

According to a third aspect, the invention relates to a method for diagnosing a renal disorder in vivo in human beings or animals, to whom nanoparticles as defined above have been administered.

According to a fourth aspect, the invention relates to a method for monitoring the therapeutic efficacy of the treatment of a renal disorder in human beings or animals to whom ultrafine nanoparticles according to the invention have been administered, preferably intravenously, said method comprising the following steps:
(i) images are captured by an appropriate imaging technique in order to visualize said nanoparticles in the kidney;
(ii) the enhancement is determined;
(iii) steps (i) and (ii) are repeated during the treatment of the subject, as many times as necessary;
(iv) the therapeutic efficacy of the treatment is deduced by comparing the change in the enhancement during the treatment.

According to a fifth aspect, the invention relates to a method for monitoring the therapeutic efficacy of a treatment of a renal disorder in human beings or animals, said method comprising the following steps:
(i) at the initiation of the treatment, ultrafine nanoparticles according to the invention, as an imaging agent, are administered to the patient,
(ii) images are captured by an appropriate imaging technique in order to visualize said nanoparticles in the kidney;
(iii) the enhancement is determined;
(iv) steps (i) and (iii) are repeated during the treatment of the subject, as many times as necessary;
(v) the therapeutic efficacy of the treatment is deduced by comparing the change in the enhancement during the treatment.

### DETAILED DESCRIPTION OF THE INVENTION

The present invention ensues from the surprising advantages demonstrated by the inventors of an administration of certain ultrafine nanoparticles, that can be used as an imaging agent, including in T1 MRI, for the diagnosis of a renal disorder, in particular renal fibrosis. All of the applications which ensue therefrom are thus related to the chemical and structural characteristics of these nanoparticles which are described below.

The invention in fact relates to nanoparticles intended to be used as an imaging agent in a method for diagnosing, in vivo, a renal disorder in human beings or animals, said particles comprising:
(i) a polyorganosiloxane (POS) matrix;
(ii) one or more chelating agents, preferably from 6 to 20 chelating agents, grafted onto the POS matrix by —Si—C— covalent bonding;
(iii) one or more metal ions chelated by one or more of the abovementioned chelating agents.

For the purposes of the invention, the term “diagnosing” and “diagnosing by imaging” can be used without distinction.

For the purposes of the invention, the term “imaging agent” is intended to mean any product or composition used in medical imaging for the purpose of artificially increasing the contrast, making it possible to visualize a particular anatomical structure (for example, certain tissues or an organ).

In one particular embodiment, the ultrafine nanoparticles according to the invention are used as an imaging agent for applications in magnetic resonance imaging (MRI), in particular in dynamic MRI (i.e. DCE for Dynamic Contrast Enhanced sequence). In this embodiment, the nanoparticles comprise at least one metal ion for T1 MRI imaging, which is particularly suitable for imaging the kidneys. MRI makes it possible in particular to obtain a particularly advantageous spatio-temporal accuracy for implementing the present invention.

For the purposes of the invention, the term “metal ion” is intended to mean any metal ion which makes it possible to confer on the imaging agent its contrast-increasing properties, that is to say its signal-increasing properties. The choice of the metal ion will be determined according to the imaging technique used.

In one particular embodiment, the metal ion is an ion of a radioactive metal, such as a cation of a radioactive metal ion Mn+, n being an integer ranging from 2 to 4, for example equal to 2, equal to 3 or equal to 4. In particular, the cation of a radioactive metal ion is a lanthanide, preferably chosen from Dy (Dysprosium), Lu (Lutecium), Gd (Gadolinium), Ho (Holmium), Eu (Europium), Tb (Terbium), Nd (Neodymium), Er (Erbium), Yb (Ytterbium) or mixtures thereof, and even more preferentially Gd. In one particular embodiment, the radioactive metal is iron (Fe) or manganese (Mn).

Preferably, at least one lanthanide cation, and including at least 50% of Gd, Dy or Ho or mixtures thereof, will be chosen as metal ion, preferably Gd, for example at least 50% of Gd, for example 100% of Gd (that is to say only Gd3+ as metal ion). In particular, Gd3+ makes it possible to obtain an optimal signal in imaging mode, in particular in MRI imaging mode.

The nanoparticles according to the invention can be used in any type of imaging, in particular one of the following imaging techniques:
(i) MRI, preferably T₁ MRI,
(ii) PET (Positron Emission Tomography) scintigraphy or SPET (Single Photon Emission Tomography) scintigraphy,
(iii) fluorescence in the visible range or in the near infrared range,
(iv) X-ray tomodensitometry.

For scintigraphy imaging, the nanoparticles comprise a radioactive isotope that can be used in scintigraphy, and preferably chosen from the group consisting of the radioactive isotopes of In, Te, Ga, Cu, Zr, Y or Lu, for example 111In, 99mTc, 68Ga, 64Cu, 89Zr, 90Y, 177Lu. The radioactive isotope is then chelated by a chelating agent grafted onto the POS matrix.

For fluorescence in the visible range, use may be made of a lanthanide chosen from Eu or Tb. The lanthanide is then chelated by a chelating agent grafted onto the POS matrix.

For fluorescence in the near infrared range, use may for example be made of an appropriate fluorophore, for example an organic fluorophore such as a cyanine, an alexa, a bodipy, or an indocyanine green (ICG). The fluorophore is advantageously grafted onto the nanoparticle, advantageously by covalent grafting using an appropriate grafting method.

For X-ray tomodensitometry, the same metal ions as for MRI can be used. Use may for example by made of a lanthanide, such as Gd.

In one particular embodiment, the nanoparticles that can be used according to the invention are characterized in that they comprise at least one metal ion for T₁ MRI imaging, and optionally at least one other metal ion suitable for one of the following imaging techniques:
(i) PET (Positron Emission Tomography) scintigraphy or SPET (Single Photon Emission Tomography) scintigraphy,
(ii) fluorescence in the visible range or in the infrared range,
(iii) X-ray tomodensitometry.

According to the invention, one or more metal ions are chelated by one or more of the chelating agents grafted onto the nanoparticles.

For the purposes of the invention, the term “chelating agent” is intended to mean a chemical substance acting as a chelate, that is to say a chemical substance which has the property of fixing positive ions in a long-lasting manner.

The nanoparticles of the invention comprise one or more chelating agents, preferably from 6 to 20, advantageously from 8 to 10 chelating agents, grafted onto the POS matrix by —Si—C covalent bonding.

According to the invention, a portion or all or of these chelating agents are intended to complex metal ions, preferably Mⁿ⁺ cations (e.g. gadolinium). Another portion of these chelating agents can be used for the complexation of endogenous cations in order to provide good compatibility with the biological media encountered. In one preferred embodiment, all the chelating agents are chelated with a metal ion, preferably Mⁿ⁺ cations (e.g. Gd³⁺).

Advantageously, the chelating agent is chosen from the following products:
the products of the group of polyamino polycarboxylic acids and derivatives thereof, and even more preferentially in the subgroup comprising: DOTA (1,4,7,10-tetraazacyclododecane-1,4,7,10-tetraacetic acid), DTPA (diethylenetriamine-pentaacetic acid), EDTA (ethylenediaminetetraacetic acid), EGTA (ethylenebis-(oxyethylenenitrilo)tetraacetic acid), BAPTA (1,2-bis-o-aminophenoxyethane-N,N,N′,N′-tetraacetic acid), NOTA (1,4,7-triazacyclononane-1,4,7-triacetic acid) and mixtures thereof;
the products of the group comprising porphyrin, chlorine, 1,10-phenanthroline, bipyridine, terpyridine, cyclam, triazacyclononane, derivatives thereof and mixtures thereof;
and mixtures thereof.

If the metal ion is a lanthanide, the chelating agent is advantageously selected from those which have lanthanide-complexing properties, in particular those of which the complexation constant log(K_{Ci}) is greater than 15, preferentially 20. As preferred examples of lanthanide-complexing chelating agents, mention may be made of those comprising a diethylenetriaminepentaacetic acid (DTPA), 1,4,7,10-tetraazacyclododecane-1,4,7,10-tetraacetic acid (DOTA), or 1,4,7-triazacyclononane-1,4,7-triacetic acid (NOTA) unit or derivatives thereof.

In one preferred embodiment, the metal ion is Gd3+ and the chelating agent is 1,4,7,10-tetraazacyclododecane-1,4,7,10-tetraacetic acid (DOTA). This combination is particularly advantageous in T1 MRI.

Advantageously, the nanoparticles are chosen such that they have a relaxivity (r1) per particle included in a range of from 50 to 5000 mM-1.s-1 and/or a weight ratio of metal ions of at least 5%, for example ranging from 5 to 50%. Preferably, the nanoparticles have a relaxivity r1 per particle included in the range of from 50 to 5000 mM-1.s-1 and/or a weight ratio of Gd3+ of at least 5%, for example ranging from 5 to 50%, advantageously ranging from 10% to 15%. The weight ratio of Gd3+ corresponds to the weight of Gd3+ relative to the weight of the nanoparticle.

The POS matrix has a diameter which may then range from 0.5 to 8 nm, advantageously from 1 to 5 nm, and may represent from 25% to 75% of the total volume of the nanoparticles. It allows in particular the grafting of the chelating agent(s).

In one particular embodiment, the nanoparticles furthermore comprise one or more functionalizing agents grafted onto the POS matrix or onto the chelating agent.

The functionalizing agent(s) can be chosen from:
one or more targeting molecules for targeting the nanoparticles. These targeting molecules make it possible to target the nanoparticles to specific tissues and/or to specific cells. They can for example target renal tissues, for example tissues of the renal cortex, the inner renal medulla or the outer renal medulla. In this way, the nanoparticles are concentrated in the area of interest without having to very significantly increase the amounts administered;
one or more fluorescent agents;
one or more hydrophilic polymers, preferably polyethylene glycol (PEG). PEG-type hydrophilic compounds make it possible to adjust the biodistribution of the nanoparticles within the body. The hydrophilic polymer(s) allow(s) greater stealth and/or neutrality of the nanoparticles. This type of coating makes it possible in particular to reduce any nephrotoxicity phenomena associated with the nanoparticles;
a combination of these agents.

The functionalizing agent(s) are grafted onto the POS matrix or onto the chelating agent. Use may be made of a conventional coupling with reactive groups present on the nanoparticles and on the functionalizing agent, optionally preceded by an activation step. The coupling reactions are known to those skilled in the art and will be chosen according to the structure of the nanoparticles and of the functional groups of the functionalizing agent(s). See, for example, “Bioconjugate Techniques”, G. T Hermanson, Académie Press, 1996, in “Fluorescent and Luminescent Probes for Biological Activity”, Second Edition, W. T. Mason, published by Académie Press, 1999. Preferably, these functionalizing agents are grafted onto the nanoparticle chelating agents.

According to one variant of the invention, the nanoparticles can be functionalized at their surface with hydrophilic compounds chosen from the group of polyols, sugars, dextrans and mixtures thereof. Glycols, advantageously PEG, are particularly preferred. According to one alternative, these hydrophilic compounds can be chosen from those which have molar masses of less than 2000 g/mol, preferably less than 800 g/mol.

The functionalizing agent(s) will be chosen according to the application envisioned.

One of the major advantages of the nanoparticles according to the invention is that they can be used as an imaging agent in imaging systems, for instance MRI, SPET scintigraphy, PET scintigraphy, fluorescence imaging, X-ray imaging.

Preferably, the nanoparticles according to the invention have a relaxivity (r1) per Mn+ ion of greater than 5 mM-1 of Mn+ ion.s-1, preferentially 10 mM-1 of Mn+ ion.s-1 for a frequency of 20 MHz. For example, they have a relaxivity (r1) per nanoparticle included in the range of from 50 to 5000 mM-1.s-1. For example, these nanoparticles have a relaxivity (r1) per Mn+ ion at 60 MHz which is greater than or equal to the relaxivity (r1) per Mn+ ion at 20 MHz. The relaxivity (r1) in question herein is a relaxivity by Mn+ ion (for example Gd3+). r1 is extracted from the following formula: 1/T1=[1/T1]water+r1[Mn+].

T1 corresponds to the longitudinal relaxation time expressed in second (s). It is the time after which the magnetization tilted in the plane during a nuclear magnetic resonance (NMR) excitation returns to its equilibrium state, by dipolar spin network interactions with the neighboring nuclei (i.e. water spins). By definition T₁ is the time interval (in milliseconds or seconds) corresponding to the recovery of 63% of the initial longitudinal magnetization. T₁ depends on the properties of the hydrogen nuclei contained in the various tissues. T₁ varies according to tissues.

[1/T₁]_{water} corresponds to the longitudinal relaxation time of water without imaging agents. This corresponds to the inverse of the longitudinal relaxation time in the absence of imaging agent, expressed in seconds⁻¹ (s⁻¹).

r₁ corresponds to the degree of relaxation, expressed in s⁻¹. This value corresponds to the return of the spin of the proton to its prior state, i.e. after excitation by RF pulse.

[Mⁿ⁺] corresponds to the concentration of metal ion, expressed in mM-1.

r1[Mn+] corresponds to the longitudinal relaxivity of the metal ion, expressed in mM-1.s-1 or in M-1.s-1. This value reflects the efficiency of a type T1 MRI imaging agent standardized with the metal ion concentration. The relaxivity r₂ is also defined by considering in the formula the relaxation time T2.

By way of indication, it is also possible to calculate the r2/r1 ratio which must be close to 1 in order to ensure the quality of type T1 agent in MRI.

In one variant, the nanoparticles have a weight ratio of T1 MRI metal ion of lanthanide type, preferably of Gd3+, of greater than or equal to 5%, for example ranging from 5% to 50%. The weight ratio of metal ions corresponds to the weight of metal ions relative to the weight of the nanoparticle.

Further details regarding these ultrafine nanoparticles, the processes for synthesizing them and the uses thereof appear in patent application WO 2011/135101, which is incorporated by way of reference, and in the publication Mignot et al (A Top-Down Synthesis Route to Ultrasmall Multifunctionnal Gd-Based Silica Nanoparticles for Theranostics Applications, Chem. Eur. J., 2013, 19, 6122-6136), which is also incorporated by way of reference.

According to the invention, the nanoparticles according to the invention have an average hydrodynamic diameter of less than 10 nm, advantageously less than 5 nm, for example ranging from 1 to 5 nm. This very small diameter allows excellent distribution of the nanoparticles in the kidneys and therefore reliable detection of a renal disorder.

The size distribution of the nanoparticles is for example measured using a commercial particle size analyzer, such as a Malvern Zêta sizer Nano-S particle size analyzer based on PCS (Photon Correlation Spectroscopy). This distribution is characterized by an average hydrodynamic diameter.

For the purposes of the invention, the term “average hydrodynamic diameter” is intended to mean the harmonic average of the diameters of the particles. A method for measuring this parameter is also described in the standard ISO 13321: 1996.

The invention is also directed toward the use of the nanoparticles according to the invention, as an imaging agent in the in vivo diagnosis of a renal disorder, for example renal fibrosis and/or renal failure, in human beings or animals. The renal disorder may also be a decrease in renal functionality, in particular decrease in tubular function and/or a decrease in glomerular filtration. The renal disorder may progress to renal fibrosis. In the most serious cases, in particular in the most serious cases of renal fibrosis, the renal disorder progresses to renal failure, in particular to chronic renal failure.

The invention is also directed toward the use of the nanoparticles according to the invention, for preparing a composition intended for the diagnosis of a renal disorder, for example renal fibrosis, for example renal failure, in human beings or animals. The renal fibrosis may be associated with involvement of the renal tubules and/or of the glomeruli.

Preferably, the composition is a pharmaceutically acceptable composition suitable for intravenous administration or administration via the airways, preferably intravenous administration.

Thus, the invention also relates to compositions comprising nanoparticles according to the invention. The compositions comprise nanoparticles according to the invention and one or more pharmaceutically acceptable excipients.

The invention is also directed toward a method for diagnosing a renal disorder in vivo in human beings or animals to whom nanoparticles as defined above have been administered. In one particular embodiment, the method comprises the steps of:
(i) capturing the images by an appropriate imaging technique in order to visualize the nanoparticles according to the invention in the kidney;
(ii) determining the enhancement;
(iii) comparing the enhancement determined in step (ii) to a reference enhancement.

In one particular embodiment, the diagnostic method is carried out in a patient to whom said nanoparticles have already been administered, that is to say that said diagnostic method does not comprise a step of administering said nanoparticles.

The comparison of the enhancement determined in step (ii) to a reference enhancement makes it possible to diagnose renal disorder, for example to diagnose renal fibrosis, for example to diagnose renal failure.

In one particular embodiment, the method comprises capturing of the images by MRI (i.e. step i) and also comprises a step (iv) of measuring the concentration of MRI imaging agent by quantitative T1 mapping. The quantitative mapping corresponds to a specific acquisition sequence determined by those skilled in the art, characterized by a T1 weighting suitable for the imaging agent, a rapidity of recording compatible with the kinetic resolution of the change in the signal and the spatial resolution quality of the imaging, and a robustness with respect to movement. The quantitative mapping can be transposed to other suitable imaging techniques.

For the purposes of the invention, the term “enhancement” or “enhancement of an imaging signal” is intended to mean the variation in the imaging signal, for example in the MRI signal, related to the arrival of the imaging agent in the tissue of interest. According to the invention, the enhancement corresponds to the variation in the imaging signal related to the arrival of the nanoparticles in the kidney, advantageously in the renal cortex, in the inner renal medulla and/or in the outer renal medulla, that is to say to the difference between the level of the signal before injection of the nanoparticles and the level of the signal after injection of the nanoparticles. The enhancement is a parameter well known to those skilled in the art. In general, the imaging devices commonly used, for example by MRI devices, provide image processing tools which make it possible to measure the enhancement of the tissue or the organ studied.

The enhancement obtained in the kidney with the imaging products conventionally used, e.g. DOTAREM in MRI, does not make it possible to distinguish a healthy kidney from a kidney suffering from a renal disorder. The inventors have demonstrated that the enhancement by the nanoparticles according to the invention in the kidney, advantageously in the renal cortex, in the inner renal medulla and/or in the outer renal medulla, makes it possible to distinguish a healthy kidney from a kidney suffering from a renal dysfuction. Thus, the nanoparticles according to the invention make it possible to diagnose a renal disorder.

The enhancement is calculated by calculating the following ratio:

r(t)=[S(t)−S0]/S0

r(t) corresponds to a degree of enhancement at a time t; S0 corresponds to the level of signal before the arrival of the imaging agent in the tissue or the organ of interest, also called precontrast signal level; S(t) corresponds to the level of signal at a time t, which corresponds to a level of signal when the imaging agent is in the tissue of interest.

According to the invention, the enhancement is determined by (i) determining the enhancement peak and/or (ii) obtaining an enhancement curve.

The “enhancement peak” corresponds to the maximum enhancement obtained after injection of the imaging agent, that is to say when the imaging signal is at its maximum. Generally, the time after injection after which the enhancement peak is obtained is common to all individuals from one and the same species. For example, in human beings, the time after injection for which the enhancement peak is attained is approximately 10 minutes, for example ranging from 5 to 15 minutes, for example ranging from 8 to 12 minutes. The time after injection after which the enhancement peak is attained for a given species can easily be determined by those skilled in the art, in particular as a function of the region of interest observed, for example the renal cortex, the renal tubules, the inner renal medulla and/or the outer renal medulla.

The “enhancement curve” corresponds to the change in the enhancement over time. By way of example, FIGS. 4, 5 and 6 correspond to enhancement curves obtained in the mouse renal cortex. The enhancement curve makes it possible to visualize the arrival and elimination over time of the imaging agent in the tissue of interest.

In one particular embodiment, the diagnosis of renal disorder is carried out by comparing the enhancement to a reference enhancement. For example, the reference enhancement corresponds to the enhancement of a healthy kidney, generally similar for all individuals of one and the same species.

According to one particular embodiment, the determination of the enhancement peak according to the invention makes it possible to determine whether the kidney is healthy or suffering from a renal disorder. Thus, a decrease in the enhancement peak of greater than 5%, for example greater than 6%, 7%, 8%, 9% or greater than 10%, for example greater than 15%, 20%, 25%, 30%, 35%, 40%, 45% or greater than 50%, relative to the reference enhancement peak, reflects a renal disorder.

According to another embodiment, the enhancement curve can also give information that can be exploited for the purpose of the invention, e.g. (i) diffusion time and (ii) elimination time of the imaging agent. Thus, the enhancement curve can also make it possible to diagnose a renal disorder. In particular, the enhancement curve is compared to a reference enhancement curve, it being possible for said reference enhancement curve to be easily determined, for example by measuring the change in the enhancement over time in a healthy individual.

One particular application of the present invention relates to the monitoring of the therapeutic efficacy of the treatment in human beings or animals, for example of a renal disorder treatment, in particular of a renal fibrosis treatment, in particular of a renal failure treatment. Thus, the invention is directed toward a method for monitoring the therapeutic efficacy of a renal disorder treatment in human beings or animals to whom nanoparticles according to the invention have been administered, in particular of a renal fibrosis treatment, in particular a renal failure treatment, said method comprising the following steps:
(i) images are captured by an appropriate imaging technique in order to visualize nanoparticles according to the invention in the kidney,
(ii) the enhancement is determined;
(iii) steps (i) and (ii) are repeated during the treatment of the subject as many times as necessary;
(iv) the therapeutic efficacy of the treatment is deduced by comparing the change in the enhancement during the treatment.
   The invention is also directed toward a method for monitoring the therapeutic efficacy of a renal disorder treatment in human beings or animals which comprises the following steps:
(i) nanoparticles according to the invention are administered to the patient, as an imaging agent,
(ii) images are captured by means of an appropriate imaging technique in order to visualize said nanoparticles in the kidney;
(iii) the enhancement is determined;
(iv) steps (i) and (iii) are repeated during the treatment of the subject, as many times as necessary;
(v) the therapeutic efficacy of the treatment is deduced by comparing the change in the enhancement during the treatment.

Thus, the change in the renal disorder over time can be monitored before, during and after the treatment of the patient.

In one particular embodiment, the enhancement is determined before, during and/or after the treatment. This makes it possible to compare the change in the enhancement during the treatment of renal disorder. According to one preferred embodiment, the change in the enhancement is compared relative to the enhancement determined before the treatment, which is then considered to be the reference enhancement. Thus, the monitoring of the enhancement makes it possible to monitor the therapeutic efficacy of the renal disorder treatment in human beings or animals.

According to one particular embodiment, the determination of the enhancement peak before, during and/or after the treatment makes it possible to determine the therapeutic efficacy of the renal disorder treatment. Thus, an increase in the enhancement peak of greater than 5%, for example greater than 6%, 7%, 8%, 9% or greater than 10%, for example greater than 15%, 20%, 25%, 30%, 35%, 40%, 45% or greater than 50%, relative to the reference enhancement peak, reflects the therapeutic efficacy of the renal disorder treatment.

The enhancement curve can also give information that can be exploited for the purpose of the invention, e.g. (i) diffusion time and (ii) elimination time of the imaging agent. Thus, the enhancement curve can also make it possible to deduce the therapeutic efficacy of the renal disorder treatment. In particular, the enhancement curve is compared to a reference enhancement curve, for example to the enhancement curve determined before treatment.

In one preferred embodiment, the nanoparticles according to the invention are visualized in the renal cortex, in the inner renal medulla and/or in the outer renal medulla. The inventors have in fact shown that the visualization of the nanoparticles according to the invention in the renal cortex enables an optimal determination of the enhancement, and thus enables a precise diagnosis of the renal disorder or a precise monitoring of the therapeutic efficacy of the renal disorder treatment.

The invention also relates to a method for treating a renal disorder in human beings or animals, comprising the following steps:
(i) administering nanoparticles according to the invention to a human being or an animal intravenously at a dose included in the range of from 5 μmol/kg to 50 μmol/kg;
(ii) capturing images by an appropriate imaging technique in order to visualize said nanoparticles in the kidney, preferably from 0 to 24 hours after the administration step;
(iii) measuring the enhancement;
(iv) when the measurement of step (iii) makes it possible to diagnose a renal disorder, introducing an appropriate treatment in the human being or the animal.

In one particular embodiment, the appropriate treatment corresponds to the administration of a sufficient amount of a medicament which makes it possible to treat the renal disorder.

In particular, the appropriate treatment can be chosen from the following treatments or a combination thereof:
standard treatments for pathological conditions inducing a renal disorder, for example diabetes, hypertension or autoimmune disease treatments;
renin-angiotensin system (RAS) inhibitors, for example angiotensin II receptor antagonist antihypertensives or inhibitors of converting enzyme responsible for angiotensin II production;
inhibitors of profibrotic cytokines, for example inhibitors of TGF-β, CTGF, PDGF, such as Pirfenidone (developed by Roche-InterMune), FG-3019 (developed by FibroGen/BMS), Fresolimumab (developed by Sanofi), THR-184 (developed by Thrasos therapeutics);
other molecules with various modes of action, for example the NOX inhibitor KGT1377831 (developed by Genkyotex), the CCR2 inhibitor CCX-140 (developed by Chemocentryx), the ALK5 inhibitor SB525334 (developed by GSK), the anti-α5β6 integrin STX-100 / Biogen Idec, or the LPA1 inhibitor BMS-986020 (developed by BMS) ;
dialysis or kidney transplantation.

In one particular embodiment, step (ii) of capturing images by an appropriate imaging technique is carried out every 3 minutes from 0 to 40 min and then every 4 h up to 24 h.

In one particular embodiment of the invention, the step of measuring the enhancement is carried out on a single kidney or on both kidneys. In particular, the enhancement can be measured for each of the kidneys.

When the metal ion is gadolinium (Gd3+), the nanoparticles according to the invention are administered intravenously at a dose of less than 0.1 mM of Gd/kg. In one particular embodiment, the nanoparticles are administered at a dose of from 0.001 to 0.1 mM of Gd/kg, advantageously from 0.01 to 0.05 mM of Gd/kg, advantageously from 0.01 to 0.02 mM of Gd/kg, advantageously at a dose of 0.017 mM of Gd/kg.

The nanoparticles according to the invention can be administered systemically, advantageously intravenously or via the airways. Patent application WO 2013/153197 describes the administration of ultrafine nanoparticles via the airways, said nanoparticles being capable of diffusing in the blood stream before being eliminated by the kidney.

In one particular embodiment of the method for monitoring the therapeutic efficacy of a renal disorder treatment according to the invention, the measurement of the enhancement makes it possible to define the Arterial Input Function (AIF). In this particular embodiment, the AIF (for example the specific AIF at kidney input) makes it possible to deduce the therapeutic efficacy of the treatment by comparing the change in the AIF in the kidney during treatment.

The AIF (or arterial input function) consists in recording the enhancement of the arterial MRI signal during the injection of the imaging agent; it is in this case measured specifically on the artery supplying the organ studied, in this case the kidney, and with a high acquisition temporal resolution of the order of one second.

### FIGURE LEGENDS

FIG. 1: Diagrammatic representation of a nanoparticle according to the invention. This nanoparticle comprises a polyorganosiloxane matrix onto which are grafted 6 chelating agents of DOTAGA type, 6 chelating agents having chelated Gd3+ gadolinium ions.

FIG. 2: Image of longitudinal SPECT/CT sections of a male c57Bl/6J mouse 15 minutes after intravenous injection of the nanoparticles obtained according to Example 1 and coupled to indium 111. K corresponds to the kidney; B corresponds to the bladder. These images show that the nanoparticles are eliminated by the kidneys and do not accumulate in the liver.

FIG. 3: Reflectance fluorescence imaging of female Swiss nude mice before (left) and after (right) the intravenous administration of the nanoparticles obtained according to Example 1 and coupled to a fluorophore of Cy5.5 type. K corresponds to the kidney. These images show that the nanoparticles are eliminated by the kidneys and do not accumulate in the liver.

FIG. 4: Comparison of the enhancement obtained by MRI after intravenous administration (i) of nanoparticles obtained according to Example 1 or (ii) of DOTAREM nanoparticles in a healthy mouse and in a UUO mouse. The results show that the DOTAREM nanoparticles cannot discriminate between a healthy kidney and a lesioned kidney, whereas a significant difference is obtained with the nanoparticles obtained according to Example 1.

FIG. 5: Time course of the measurement of the enhancement in the renal cortex by MRI after intravenous administration of DOTAREM nanoparticles (10 mM). The curves obtained show that the enhancement profile is similar between UUO kidney (lesioned kidney) and reference kidney (healthy kidney). The results show that the DOTAREM nanoparticles cannot discriminate between a healthy kidney and a lesioned kidney.

FIG. 6: Time course of the measurement of the enhancement in the renal cortex by MRI after intravenous administration of nanoparticles obtained according to Example 1 (5 mM). The curves obtained show that the enhancement profile is significantly different between UUO kidney (lesioned kidney) and reference kidney (healthy kidney). The results show that the nanoparticles obtained according to Example 1 can discriminate between a healthy kidney and a lesioned kidney.

FIG. 7: Images obtained for visualization of nanoparticles obtained according to Example 1 (AGuIX nanoparticles) in the kidneys by LIBS (Laser-Induced Breakdown Spectroscopy). LIBS makes it possible to visualize the gadolinium atoms originating from the AGuIX nanoparticles, represented in green, and the sodium atoms (in red) uniformly distributed in the tissue. The results show that the deficient kidney does not retain the nanoparticles or retains few of said nanoparticles, which is in line with the results obtained by MRI in vivo.

FIG. 8: Histological images obtained by confocal photon microscopy making it possible to visualize according to the invention grafted with a Cy5.5 chromophore (i.e. AGuIX nanoparticles grafted with a Cy5.5 chromophore, in red) in renal tubular cells. The nanoparticles are accumulated in the renal tubules, a transient step prior to elimination of said nanoparticles.

FIG. 9: Images obtained by dynamic MRI of the kidneys of a mouse (1 UUO kidney and 1 normal kidney) before and after injection of nanoparticles according to the invention. The results show that the deficient kidney does not retain the nanoparticles according to the invention or retains few of said nanoparticles. Thus, the images show that the enhancement of the renal cortex of the reference kidney is much greater than in the renal cortext of the UUO kidney.

FIG. 10A: Time course of the measurement of the enhancement in the renal cortex by high time resolution DCE MRI and processing of the data by the Mann-Whitney improved 3TP biostatistics method after intravenous administration of DOTAREM nanoparticles (10 mM). The curves obtained show that the enhancement profile is similar between UUO kidney (lesioned kidney) and reference kidney (healthy kidney). The results show that the DOTAREM nanoparticles cannot discriminate between a healthy kidney and a lesioned kidney.

FIG. 10B: Time course of the measurement of the enhancement in the renal cortex by high time resolution DCE MRI and processing of the data by the Mann Whitney improved 3TP biostatistics method after intravenous administration of nanoparticles obtained according to Example 1 (5 mM). The curves obtained show that the enhancement profile is significantly different between UUO kidney (lesioned kidney) and reference kidney (healthy kidney). The results show that the nanoparticles obtained according to Example 1 can discriminate between a healthy kidney and a lesioned kidney.

FIG. 11: Monitoring, by optical imaging, as a function of time, of the AGUIX-Cyanin 5 agent contrast uptake by the lesioned (UUO)kidney compared with the contralateral non-lesioned kidney.

FIG. 12: Profile of the arterial input function AIF obtained with Dotarem.

FIG. 13: Profile of the arterial input function AIF obtained with AGUIX.

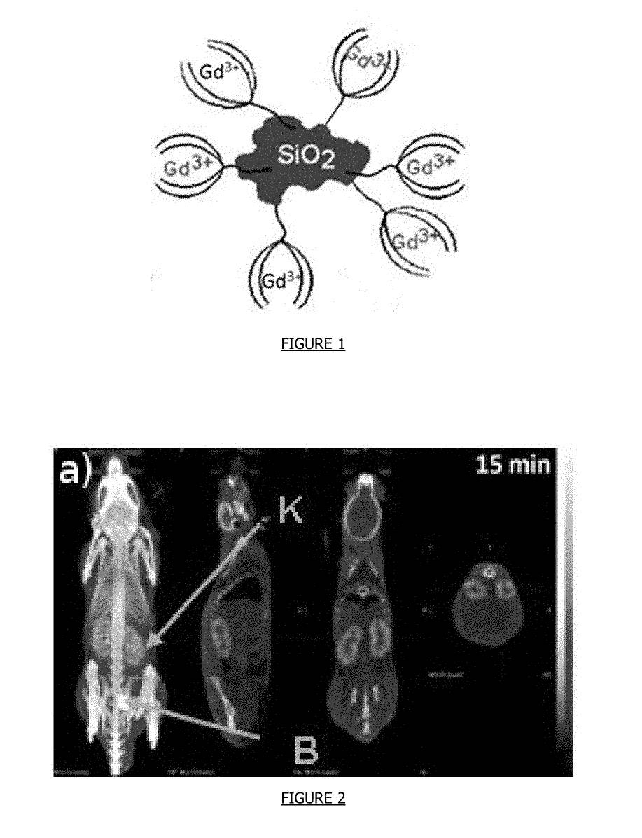

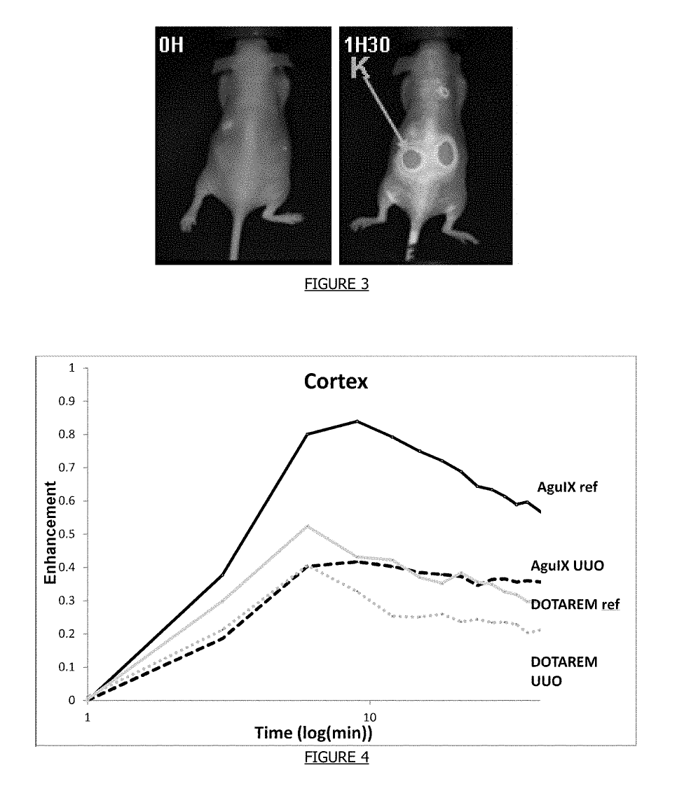

### EXAMPLES

### Example 1: Preparation of DOTAGA-Type Nanoparticles

### Nanoparticle Synthesis

A solution was prepared by dissolving 167.3 g of [GdCl₃, 6 H₂O] in 3 l of diethylene glycol (DEG) at ambient temperature. The mixture was then stirred for 3 hours at 140° C. 44.5 ml of 10M sodium hydroxide were then added, then the mixture was heated for 5 hours with stirring at 180° C. in order to obtain solution A. The gadolinium oxide cores obtained in solution A have a hydrodynamic diameter of 1.7±0.5 nm.

The polysiloxane layer was obtained by a sol-gel process (i.e. by condensation hydrolysis reactions under basic conditions obtained by adding organosilane precursors). Firstly, a first solution containing 1.6 l of DEG, 51.4 ml of tetraethoxysilane (TEOS) and 80.6 ml of aminopropyltriethoxysilane (APTES) were slowly added (i.e. over the course of 96 hours) to solution A at a temperature of 40° C. One hour after the addition of the first solution, a second solution containing 190 ml of DEG, 43.1 ml of water and 6.9 ml of triethylamine (TEA) was added with stirring for 96 hours at 40° C. At the end of the reaction, the mixture was left to stir for 72 hours at 40° C. and then for 12 hours at ambient temperature in order to obtain solution B. The gadolinium oxide cores covered with polysiloxane of solution B have a hydrodynamic diameter of 2.6±1.0 nm.

163.7 g of anhydrous DOTAGA were then added to solution B. The resulting solution was left to stir for 72 hours at ambient temperature in order to obtain solution C which contains nanoparticles comprising a gadolinium oxide core covered with polysiloxane, onto which nanoparticles DOTAGA groups are grafted.

### Purification

17.5 l of acetone were added to solution C in order to precipitate the nanoparticles, which were recovered by filtration under vacuum. The nanoparticles were then redispersed in water and placed at pH 2 for 1 hour. The remaining acetone was evaporated off. The nanoparticles were then purified by tangential filtration on a 5 kDa membrane, before the addition of sodium hydroxide so as to obtain a pH of 7.4. Passage through water and purification in water led to the dissolution of the gadolinium oxide core. This dissolution of the gadolinium oxide core is promoted by the presence of the chelating groups grafted onto the polysiloxane matrix of the nanoparticle which complex the gadolinium released by the dissolution of the core. Once the core was dissolved, the polysiloxane shell fragmented into ultrafine nanoparticles consisting of a polysiloxane matrix onto which are grafted DOTAGAs which chelated gadolinium. The solution thus obtained was then filtered twice through a 1.2 μm filter and then through a 0.2 μm filter in order to remove the bulkiest impurities. The nanoparticles thus obtained have a hydrodynamic diameter of 2.2±1.0 nm, a longitudinal relaxivity of 11.5 mmol⁻¹.L.s⁻¹ at 60 MHz and at 37° C. Approximately 50 grams of nanoparticles were obtained at the end of the synthesis.

### Example 2: Elimination of the Nanoparticles by the Kidney

LIBS (laser induced breakdown spectroscopy) elemental imaging protocol

The nanoparticles obtained according to example 1 were administered intravenously to anesthetized mice (8 mg/mouse). Said mice were then sacrificed at 15 min and at 1 h 30 in order to allow the kidneys to be removed.

The kidneys were perfused and fixed in a 2% glutaraldehyde buffer prepared in a 0.1M sodium cacodylate solution (pH 7.4), overnight at 4° C.

The kidneys were then rinsed in a 0.2M sodium cacodylate solution (pH 7.4).

After fixing, the kidneys were dehydrated by means of various ethanol baths of increasing concentration from 30% to 100% of ethanol as follows: 30%, 50%, 70%, 80%, 95%, 100%, 100%, 100%. Each bath lasted 30 min.

The kidneys were then immersed in a solution of propylene oxide/100% ethanol (1:1) for 30 min, then in 2 propylene oxide baths (each 30 min).

During the impregnation, the kidneys were immersed in various baths of propylene oxide and of EPON resin for 4 hours overnight according to the ratios (2:1), (1:1), (1:2), (0:1), (0:1).

The kidneys were then immersed in a mixture of freshly prepared EPON containing the curing agent and placed for study at 56° C. for 72 h.

The sample can then be prepared for the LIBS analysis by cutting and polishing the surface of the kidney.

The results are presented in FIGS. 7 and 8.

Conclusion: the deficient kidney only very slightly retains the nanoparticles, contrary to the functional kidney which retains the nanoparticles.

### Example 3: Measurement of the Enhancement in the Kidney by MRI: Comparison Between Nanoparticles According to the Invention (AGuIX Nanoparticles) and the Prior Art Nanoparticles (DOTAREM)

The images were acquired with a 7T 300 MHz spectrometer imager, equipped with a linear 1H radiofrequency coil dedicated to mice (Bruker, Karlsruhe, Germany).

The dynamic MRI studies of capture and release of the nanoparticles in the tissues were carried out in vivo and made it possible to determine the change in enhancement in the kidney.

All the manipulations of the animals were carried out in compliance with the institutional animal protocol guidelines in place at Paris Descartes University, submission CEEA34.JS.142.1 and approved by the Institute's animal research committee, and also the ethics protocols of the company regarding mouse inductions. The relaxivities in solutions were measurement beforehand at 7T in order to prepare the final injection concentration.

8-week-old female mice of BalbC/JRJ type (n=6) were UUO-treated at just one kidney, i.e. each mouse comprises one lesioned kidney (UUO kidney) and one healthy kidney (reference kidney). The mice were anesthetized by inhalation of isoflurane (1.5% of air/O2 0.5/ 0.25 l/min) and placed in a dedicated containment cradle. The physiological respiratory parameters were measured throughout the MRI studies using a nonmagnetic respiratory sensor system (the company SAM, US) and the body temperature was maintained by heating the cradle to the core.

Two solutions were prepared:
Solution according to the invention: 100 μl of imaging agent dissolved in a 0.9% saline solution, with a 5 mM concentration of AguIX imaging agent (i.e. the nanoparticles obtained according to Example 1).
Solution according to the prior art: 100 μl of imaging agent dissolved in a 0.9% saline solution, with a 10 mM concentration of DOTAREM imaging agent.

The solutions were administered to mice intravenously via the caudal vein by means of a catheter (specifically developed for nonmagnetic use; calibrated with known dead volume), while the mouse was in the scanner.

For the reproducibility studies, 100 μl of the solution of AGuIX nanoparticles at 5 mM was administered to the 6 mice (the healthy kidney being the reference kidney or ref., and the lesioned kidney being the UUO kidney)

The original acquisition protocol was developed with the Paravision 5.1 software.

A dynamic contrast enhanced DCE sequence was recorded using a specific sequence eliminating the free artefacts of movement in post treatment with a TR of 100 ms and a TE of 4 ms and a tilt angle of 80° in order to ensure T1 MRI weighting; a field of view (FOV) of 3 cm×3 cm and a final matrix of 256 by 256 points. 4 sections 1 mm thick were chosen, which gives a planar spatial resolution of 117 μm×117 μm. The total time of one acquisition per image was about 3 min 14 sec. Finally, an extended version of the sequence based on the repetition of the previous sequence was used for the dynamic monitoring in order to obtain the same time resolution in a cycle time of from 40 min to 4 h.

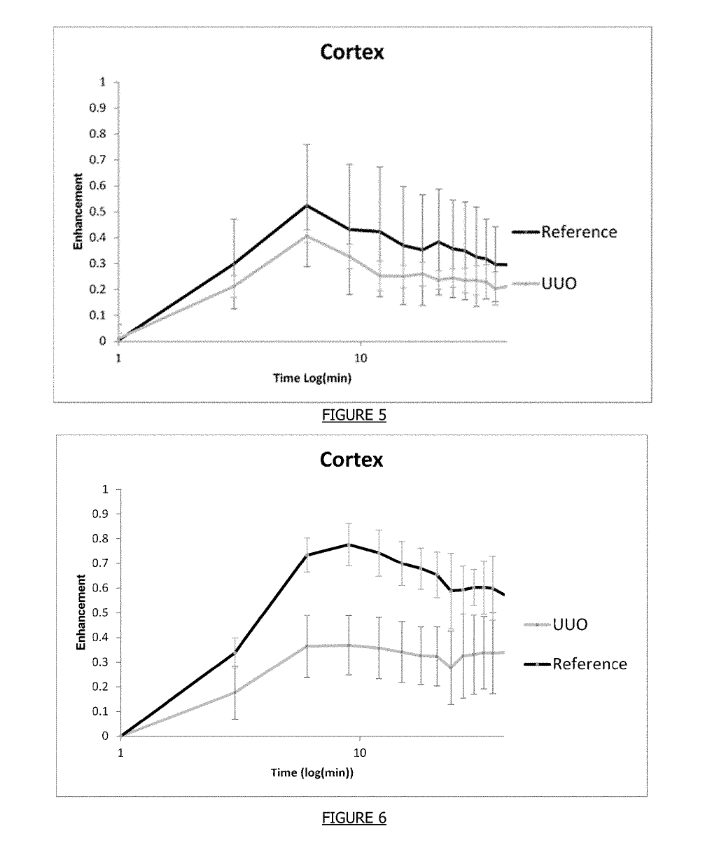

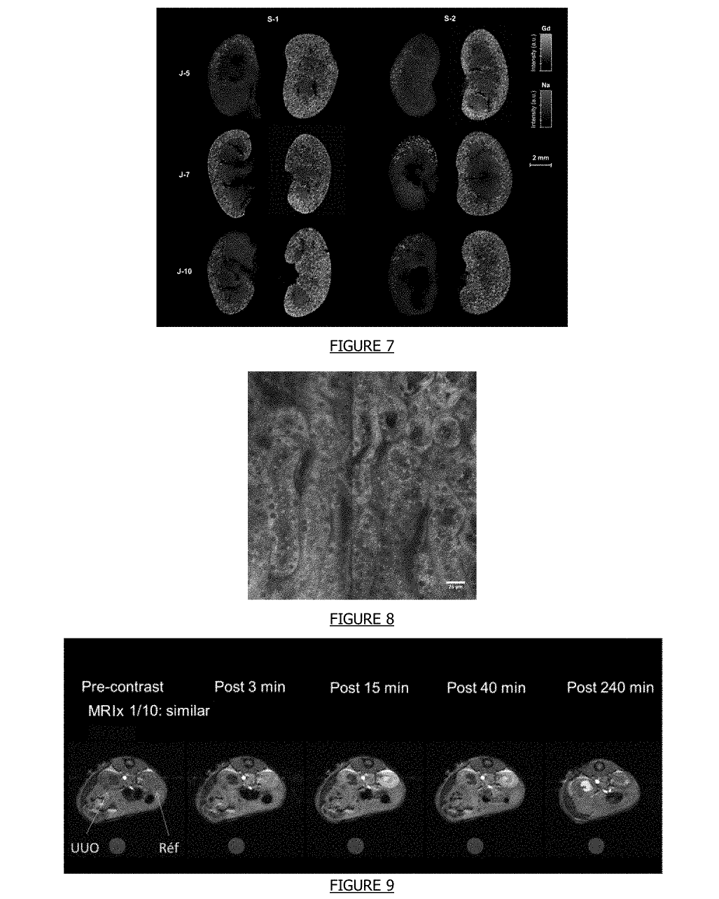

Ater the image acquisition, image processing was carried out by delimiting renal regions of interest in the cortical zones, medulla zones, or the like. The intensities obtained in the cortical zone were reported on an Excel file and the enhancement (S₀−S)/S₀ was reported on a graph as a function of time (min) at which the image was acquired. The results are presented in FIGS. 4 to 6 and FIG. 10.

In the figures, the term “SHAM” corresponds to a batch of non-UUO mice, of the same genetic background. The term “contra UUO” corresponds to the non-pathological contralateral kidney of the UUO mice which serves as a healthy kidney reference. In the interests of being rigorous, in order to verify that the contralateral kidney was not undergoing any overcompensation, a batch of non-UUO-induced control mice was studied (SHAM mice), confirming that the results obtained with the SHAM mice kidneys are identical to the results obtained with the UUO contralateral kidney.

Conclusion: The nanoparticles according to the invention make it possible to significantly differentiate renal functionality between a healthy kidney and an obstructed kidney. DOTAREM does not make it possible to significantly differentiate renal functionality of a healthy kidney and of an obstructed kidney.

### Example 4: High Temporal Resolution Perfusion MRI Method for the Characterization of a Pathological Renal Condition: Comparison Between Nanoparticles According to the Invention (AGuIX Nanoparticles) and the Prior Art Nanoparticles (DOTAREM)

The DCE ‘Dynamic Contrast Enhanced’ perfusion MRI method conventionally used in MRI imaging of renal function consists in acquiring MRI image kinetics with a temporal resolution of 1 s to 10 s in the literature. It provides kinetic profiles of signal enhancement corresponding to the immediate arrival of the imaging agent in the tissue, in particular for probing the first glomerular pass. These dynamic curves are digitally processed using pharmacokinetic models and provide information regarding the microvascularization, such as blood flows, blood volume fractions, vascular permeability, extracellular volume, etc. However, these results lack reproducibility and require personalized modeling for which there is currently no consensus. Currently, there is ultimately no ideal contrast agent for evaluating renal function. A more appropriate match between the physiological phenomenon of interest and the pharmacokinetic characteristics of the agent must be found. In our case, we propose to develop the high temporal resolution DCE MRI method with the AGuIX agent for improving the diagnosis of a renal disorder on the ureteral obstruction UUO model in mice.

The UUO model is induced by clamping the right ureter of female BalbC/JRJ mice according to a biochemically characterized protocol from the literature. All the manipulations of the animals were carried out in compliance with the institutional animal protocol guidelines described in Example 3.

8-week-old female mice of BalbC/JRJ type (n=6) were UUO-treated at the level of just one kidney, i.e. each mouse comprises one lesioned kidney (UUO kidney) and one healthy kidney (reference kidney). A batch of sham mice makes it possible to verify the validity of the contralateral reference with the MRI method developed. 2 batches of n=6 mice and n=6 shams were studied by dynamic MRI 3 days after induction.

The DCE MRI acquisition protocol consists in injecting the two types of imaging agents: reference DOTAREM and AGuIX, namely:

Solution according to the invention: 100 μl of imaging agent dissolved in a 0.9% saline solution, with a 5 mM concentration of AGuIX imaging agent.

Prior art solution: 100 μl of imaging agent dissolved in a 0.9% saline solution with 10 mM concentration of DOTAREM imaging agent.
i) The administration protocol is that of Example 3.
ii) The MRI acquisition protocol itself consists in recording sequential images by means of the T1-weighted FLASH MRI sequence (TR=TE=2.2 ms, spatial resolution 117×310 μm²), temporal resolution 2.5 s for 10 min with 20 s of precontrast. FIG. 10 shows the enhancement profiles for the 2 agents, in the UUO lesioned kidney and the reference contralateral kidney. T1 mapping in order to verify the concentration of Gd MRI agent is recorded.
iii) The digital processing method derived from method termed 3TP, three time Points, consists in defining 3 main points: basic reference intensity, point of maximum intensity of enhancement, final clearance time, and calculating the slopes of capture and of release of the agent. Next, a Mann-Whitney biostatistical study is applied to the slope parameters calculated for each of the 6 mice, and for each imaging agent.

Averaged collective method resulting in the value of T1max:
a) Searching for the time T corresponding to the maximum in the overall mean of the DCE MRI intensities.
b) Applying this max to each enhancement profile as Tmax.
c) Taking the mean of 6 enhancement values around the indexed value.

Conclusion: The diagnosis provided by the high temporal resolution MRI method on the UUO model is better with the AguIX agent since the differential in the impact on the DCE MRI signal linked to the perfusion, between the healthy and pathological kidney condition that can be obtained is substantially greater, the decrease is 350% or a factor of 3.5 with AguiX, whereas it is only 100% or a factor of 2 with DOTAREM. This higher differential obtained with AGuIX enables a better diagnosis of the pathological renal condition with the AGuIX imaging agent.

### Example 5: AGuIX Kinetics in Optical Imaging on UUO-Treated Mice

8-week-old female mice of BalbC/JRJ type (n=6) were UUO-treated as described in Example 3.

Solution prepared: 100 μl of imaging agent dissolved in a saline solution of sodium chloride at 0.9%, with a 10 mM concentration of AguIX imaging agent coupled to a cyanine 5.5, Cy5.5 fluorophore (AguIX-Cy5.5).

After anesthesia of the mice by injection of ketamine, a catheter was placed in the tail vein and the agent was injected. The dynamic monitoring was carried out with the Fluobeam camera at the level of the kidney. The wavelengths and the parameters that were used are: excitation 680 nm and emission >700 nm; exposure time: 50 ms; gap between images: 2 sec. The data were processed with ImageJ with an ROI of the same size for all the kidneys then by using the following calculation

enhancement=(RawIntDen(t)−RawIntDen(t0))/RawIntDen(t0)

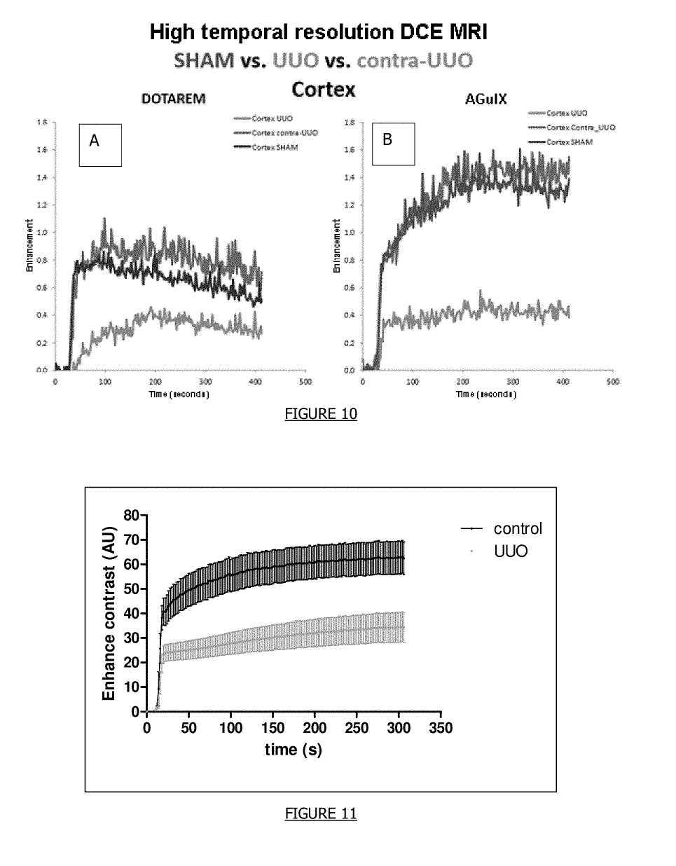

FIG. 11 represents the kinetics of arrival of the imaging agent in the kidneys for the first five minutes (n=6 kidneys per condition).

Conclusion: the results obtained confirm the MRI results previously obtained with the AGuIX agent not coupled to cyanine 5.5. The results also show that similar results can be obtained by two different imaging modes with the AGUIX agent comprising the required imaging functionality. It is possible to diagnose the renal UUO pathological condition by also using the in vivo optical imaging in dynamic acquisition method as has been developed in MRI, known to be the quantitative method of reference in perfusion.

### Example 6: Diagnosis of a Therapy Against a Dysimmune Pathological Renal Condition by Molecular MRI with AguIX-Monitoring of Therapeutic Efficacy

MRI model and image processing

The high temporal resolution DCE perfusion method with a period 2.5 s for 10 min, with injection of the imaging agent as a 100 μl bolus at 5 nM (method described in Example 4) was applied on another model of glomerular and tubular renal disorder, i.e. model induced by injection of anti-gbm antibodies in the literature.

The DCE MRI acquisition protocol consists in injecting the two types of agents: reference DOTAREM or AGuIX, namely:

solution according to the invention: 100 μl of imaging agent dissolved in a 0.9% saline solution, with a 5 mM concentration of AguIX imaging agent, and

solution according to the prior art: 100 μl of imaging agent dissolved in a 0.9% saline solution, with a 10 mM concentration of DOTAREM imaging agent.

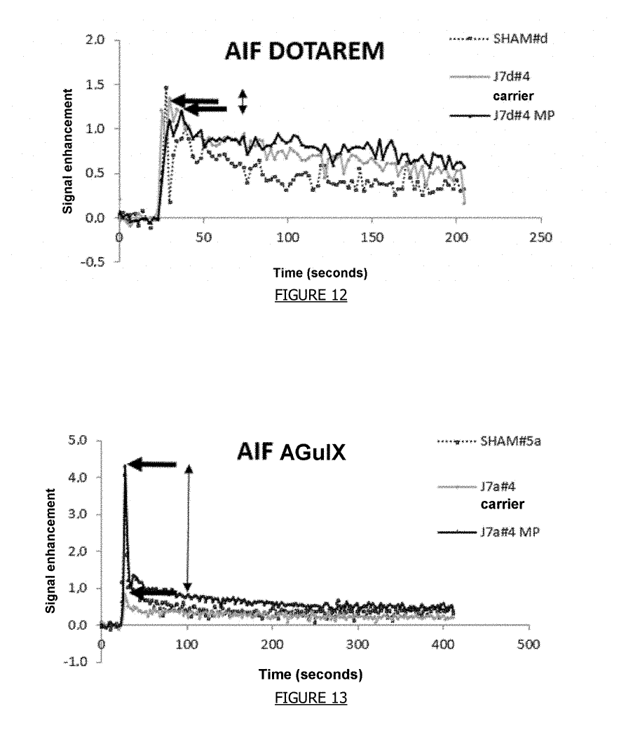

The MRI acquisition protocol itself consists in recording sequential images by means of the T1-weighted FLASH MRI sequence (TR=TE=2.2 ms, spatial resolution 117×310 μm²), temporal resolution 2.5 s approximately 120 s with 20 s of precontrast. FIG. 12 shows the enhancement profiles measured on the renal artery on the dynamic images recorded, AIF, for the 2 agents.
i) The data processing corresponds to comparing the ratio of the maximum enhancement between the AIF data from carrier pathological kidneys and pathological kidneys after therapy.

The glomerular and tubular renal disorder model is obtained by IV injection of Probetex antibody serum PTX-001S in 6-to-8-week old male CBA mice, targeting the glomerular basal membrane and inducing a lesion of the glomerulus, the capsule of which becomes crescent shaped and atrophies, cysts appear, an inflammation and biochemical markers for proteinuria (increase in albumin/creatinine level) and increase of myofibroblasts by alpha SMA assay.

The therapy was obtained using methyl prednisolone hemisuccinate anti-inflammatory injected intraperitoneally at 25 mg/kg twice a week.

The imaging was carried out on d7 and d14 post-induction of the pathological condition.

The pathological model is characterized and diagnosed by the high time resolution DCE perfusion method described in Example 4.

An additional parameter corresponding to the arrival of the imaging tracer in the organ, i.e. arterial input function AIF, was also recorded. Measured at the input of the organ studied, the AIF itself at the input of the kidney is an indicator of the functionality. The profile is recorded on the anatomical MRI image at the level of the renal arterial point on the dynamic images acquired at DCE high temporal resolution proposed.

The AIF parameter made it possible in particular to evaluate the monitoring of the efficacy of a therapy.

Results: FIGS. 12 and 13 show that the efficacy of a therapy diagnosed by in vivo MRI imaging is observed only with the AGuIX agent by measuring the AIF parameter at the kidney input. Specifically, the AIF is identical with DOTAREM between the control mouse (carrier mouse) pool and the mice treated with the anti-gbm antibody (MP mice), whereas it is different, and much higher with the AGuIX agent in the mice treated with the anti-gbm antibody (MP mice).

1. Nanoparticles for diagnosing, in vivo, a renal disorder in human beings or animals, said nanoparticles comprising:
(i) a polyorganosiloxane (POS) matrix;
(ii) one or more chelating agents grafted onto the POS matrix by —Si—C— covalent bonding;
(iii) one or more metal ions chelated by one or more of the chelating agents.

2. The nanoparticles of claim 1, wherein said nanoparticles have an average hydrodynamic diameter of less than 10 nm.

3. The nanoparticles of claim 1, wherein said nanoparticles comprise at least one metal ion for T₁ MRI imaging.

4. The nanoparticles of claim 1, wherein said nanoparticles have a relaxivity (r₁) per particle ranging from 50 to 5000 mM⁻¹.s⁻¹ and/or a weight ratio of metal ion of at least 5%, for example ranging from 5% to 50%.

5. The nanoparticles claim 1, wherein the metal ion is a radioactive metal ion.

6. The nanoparticles of claim 5, wherein the radioactive metal ion is a lanthanide selected from: Dy, Lu, Gd, Ho, Eu, Tb, Nd, Er, Yb and mixtures thereof.

7. The nanoparticles of claim 6, wherein the chelating agent is a lanthanide-complexing agent is selected from: diethylenetriaminepentaacetic acid (DTPA), 1,4,7,10-tetraazacyclododecane-1,4,7,10-tetraacetic acid (DOTA), 1,4,7-triazacyclononane-1,4,7-triacetic acid (NOTA) efand derivatives thereof.

8. The nanoparticles of claim 1, wherein the metal ion is Gd³⁺ and the chelating agent is 1,4,7,10-tetraazacyclododecane-1,4,7,10-tetraacetic acid (DOTA).

9. The nanoparticles of claim 1, further comprising a functionalizing agent selected from: a fluorescent agent, a polyethylene glycol (PEG) and a targeting molecule for targeting the nanoparticles, wherein the functionalizing agent is grafted onto the POS matrix or onto the chelating agent.

10. The nanoparticles of claim 1, wherein the renal disorder is renal fibrosis and/or renal failure.

11. A composition comprising the nanoparticles of claim 1, which composition is intended for use as an imaging agent in the in vivo diagnosis of a renal disorder in human beings or animals.

12. A composition comprising the nanoparticles of claim 1, which composition is intended for use in the diagnosis of a renal disorder in humans or animals.

13. A method for diagnosing a renal disorder in vivo in a human being or an animal to whom the nanoparticles of claim 1 have been administered, the method comprising the steps of
(i) capturing one or more images by an appropriate imaging technique in order to visualize said nanoparticles in at least one kidney of the human being or the animal to whom the nanoparticles of claim 1 have been administered;
(ii) determining an enhancement of the images; and
(iii) comparing the enhancement obtained in step (ii) to a reference enhancement to diagnose the renal disorder in vivo in the human being or the animal.

14. A method for monitoring the therapeutic efficacy of the renal disorder treatment in a human being or an animal to whom the nanoparticles of claim 1 have been administered, said method comprising the steps of:
(i) capturing one or more images by an appropriate imaging technique in order to visualize said nanoparticles at least one kidney of the human being or the animal to whom the nanoparticles of claim 1 have been administered,
(ii) determining an enhancement of the images;
(iii) repeating steps (i) and (ii) during the treatment of the human being or the animal one or more times;
(iv) deducing the therapeutic efficacy of the treatment by comparing the change in the enhancement during the treatment.

15. A method for monitoring the therapeutic efficacy of a renal disorder treatment in a human being or an animal, said method comprising the steps of:
(i) administering the nanoparticles of claim 1 to the human being or the animal, as an imaging agent,
(ii) capturing one or more images by an appropriate imaging technique in order to visualize said nanoparticles in at least one kidney of the human being or the animal;
(iii) determining an enhancement of the images;
(iv) repeating steps (i) and (iii) during the treatment of the human being or the animal one or more times;
(v) deducing the therapeutic efficacy of the treatment by comparing the change in the enhancement during the treatment.

16. The nanoparticles of claim 1, wherein said nanoparticles comprise from 6 to 20 chelating agents.

17. The nanoparticles of claim 2, wherein said average hydrodynamic diameter ranges from 1 to 5 nm.

18. The nanoparticles of claim 4, wherein said weight ratio ranges from 5% to 50%.

19. The nanoparticles of claim 5, wherein said radioactive metal ion is a cation of a radioactive metal ion Mⁿ⁺, n being an integer ranging from 2 to 4.
